(19) United States
(12) Patent Application Publication
(10) Pub. No.: US 2004/0115239 A1
(43) Pub. Date: Jun. 17, 2004

(54) Engineering of material surfaces

(76) Inventors: Venkatram P. Shastri, Lower Gwynedd, PA (US); I-Wei Chen, Swartmore, PA (US); Hoon Choi, Bryn Mawr, PA (US); Anna Marie Lipski, Philadelphia, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/668,484

(22) Filed: Sep. 22, 2003

Related U.S. Application Data
(63) Continuation-in-part of application No. 10/427,242, filed on May 1, 2003.
(60) Provisional application No. 60/411,871, filed on Sep. 20, 2002.

(51) Int. Cl. A61F 2/00

(52) U.S. Cl. 424/423

(57) ABSTRACT

The invention provides a device having a surface and a functional layer associated with the surface, where the functional layer includes particles having a structure substituted with a functional group, where the functional group is adapted to modify a property of the device, the device is sufficiently biocompatible for application to a multicellular organism and the particles have an average diameter of about 5 nm to about 10 microns.

### BACKGROUND OF THE INVENTION

1. Field of Invention

This invention relates to providing surfaces that are characterized by a well-defined, small-scale topography and by a chemical functionality that accommodates attachment of biomolecules to surfaces to create modified surfaces that are conducive to the attachment, migration and growth of cells. Further, this invention relates to providing implantable surfaces.

2. Description of Related Art

For a variety of biomedical applications, it is important to provide a surface that is not only cytocompatible but also conducive to cell migration, proliferation and differentiation. A requirement of a successful orthopedic implant, for example, is an ability to support new osseous tissue formation into and around the implant. Conversely, poor integration of an implant with surrounding bone, leading to osteolysis, is a significant mode of failure for many existing orthopedic implants.

More generally, a primary goal of tissue engineering (TE) is to foster the synthesis or regeneration of tissues, for transplantation or in situ growth, by means of defined structures or “scaffolds” that can support the development of cells, which may be seeded or which may migrate into the structure, forming new, functional tissue. To these ends, a greater understanding of the factors governing cellular processes has opened the way, in principle, to manipulating those processes via surface-localized, bioactive polypeptides or encoding polynucleotides. In this regard, an implant also is a potentially attractive vehicle for delivering biomolecules that can direct tissue formation or otherwise affect proximal tissue(s) in desired ways.

Coating an implant or a scaffold surface is one way to condition that surface to accommodate cell attachment and subsequent development, optionally under the influence of bioactive molecules also localized on the surface. Conventional coating techniques are poorly defined at the sub-micron level, however, and may not provide a suitable bio-mimetic interface for attaching cells. Furthermore, known coatings typically yield a surface lacking chemical reactivity that is needed for the immobilization and presentation of bioactive molecules.

U.S. Pat. No. 4,243,692 to Scholze et al., discloses the use of silicic acid heteropolycondensates as coating compositions in the culture of living cells. Scholze et al., disclose that a substituted silane, a functional silane, and a hydrolyzable silicic acid derivative are simultaneously condensed to produce the heteropolycondensate. The process of this patent does not involve the application of silicon dioxide particles to a substrate.

U.S. Pat. No. 5,814,550 to Wolcott discloses coating of surfaces using an aqueous solution of colloidal silica to provide surfaces which are conducive to the growth of primary cells. Wolcott does not disclose coating of surfaces using modified or functionalized colloidal silica.

Coating of surfaces using silicon dioxide is described by W. Stober, A. Fink, and E. Bohn, “Controlled Growth of Monodisperse Silica Spheres in the Micron Size Range,” J. Colloid Interface Sci., 26, 62-69(1968); M. Atik, P. De LimaNeto, L. A. Avaca, M. A. Aegerter, J. Zarzycki, “Protection of 316L Stainless Steel Against Corrosion by SiO₂ Coatings,” J. Mater. Sci. Lett. 13 1081-1085 (1994); K. Yoshida, K. Kamada, K. Sato, R. Hatada, K. Baba, M. Atsuta, “Thin Sol-Gel-Derived Silica Coatings on Dental Pure Titanium Casting,” J. Biomed. Mater. Res. 48: 778-785 (1999); and D. C. L. Vasconcelos, J. A. N. Carvalho, M. Mantel, W. L. Vasconcelos, “Corrosion Resistance of Stainless Steel Coated with Sol-Gel Silica,” 273 135-139 (2000). These references do not disclose coating of surfaces using modified or functionalized colloidal silica.

Other related technologies and background are described in the following publications: E. P. Plueddemann, “Silane Coupling Agents,” Plenum Press, New York, Chapter 3, 49-73 (1982) and K. C. Vrancken, K. Possemiers, P. Van Der Voort, E. F. Vansant, “Surface Modification of Silica Gel with Aminoorganosilanes,” Colloids and Surfaces, 98 235-241 (1995).

Polymeric colloidal particles are typically prepared by one of the three methods.

In the method of emulsification-solvent evaporation, the polymer is dissolved in chlorinated hydrocarbon (organic solvent) such as methylene chloride or chloroform as disclosed by Wise, Donald L. ed., Handbook of Pharmaceutical Controlled Release Technology, Marcel Dekker Incorporated, New York, N.Y., pages 329-344 (2000). The polymer solution is then mechanically dispersed in an aqueous solution containing a polymeric surfactant, such as polyvinyl alcohol (PVA) or carboxymethoxycellulose (CMC), by homogenization or ultrasonication to form a microemulsion. The thermodynamically unstable microemulsion is stabilized by the presence of PVA. The organic solvent is then evaporated and the colloids (and/or NPs) collected by centrifugation to remove the excess PVA and then resuspended in a solution of interest.

Niwa et al. have developed a method to produce polymeric colloidal particles by first dissolving the polymer in a mixture of chlorinated hydrocarbon such as methylene chloride and acetone, and then pouring this solution into a aqueous phase containing PVA with mechanical stirring. (See Controlled Rel., (25), 89-98 (1993)). Acetone is added to enhance the diffusion of the methylene chloride solvent into the water phase. Like the solvent evaporation approach the organic solvent is evaporated and the colloids are separated from the PVA phase by centrifugation. Their approach is called spontaneous emulsification solvent diffusion (SESD).

Murakami et al. have reported a modification of the SESD procedure that relies on the gelation of the PVA phase around the emulsion droplets for stabilization of the colloids as they form in solution. (See Intl. J. Pharm., (187), 143-152 (1999)). In this approach, to control and restrict the gelation of PVA to the surface of the emulsion droplet, alcohol (ethanol or methanol), which is a solvent for PVA but a non-solvent for the polymer was used. The mechanism of colloid formation is again dependent on the presence of the polymeric emulsifier, PVA. This method yields colloids of mean diameter of above 260 nm.

Despite the foregoing developments, there is still a need in the art for cytocompatible surfaces that are beneficial to cell migration, proliferation and differentiation and for surfaces that possess chemical reactivity that is needed for the immobilization and presentation of bioactive molecules.

All references cited herein are incorporated herein by reference in their entireties.

### BRIEF SUMMARY OF THE INVENTION

Accordingly, the invention provides a device comprising a surface and a functional layer associated with the surface, wherein the functional layer comprises particles having a structure substituted with a functional group, wherein the functional group is adapted to modify a property of the device, the device is sufficiently biocompatible for application to a multicellular organism and the particles have an average diameter of about 5 nm to about 10 microns.

In certain embodiments, the device is an implantable device. In other embodiments, the device is a drug delivery device. In certain embodiments, the device is an outer surface contacting device adapted to contact an outer surface of the multicellular organism.

In certain embodiments, the multicellular organism is a human.

Further provided is a method of modifying a surface, said method comprising providing on the surface a functional layer comprising particles having a structure substituted with a functional group and/or associated with a functional moiety such that the functional layer modifies a property of the surface to provide a modified surface, wherein the modified surface is sufficiently biocompatible for application to a multicellular organism and the particles have an average diameter of about 5 nm to about 10 microns.

In certain embodiments, the property is adhesion, friction, wettability, texture or roughness.

In certain embodiments, the functional layer modifies a reaction to the surface of a cell of the multicellular organism.

In certain embodiments, the functional layer modifies a reaction to the surface of a tissue of the multicellular organism.

In certain embodiments, the modified surface transfects with genomic material adjacent cells and tissue.

In certain embodiments, the modified surface delivers bioactive agents to adjacent cells and tissue.

In certain embodiments, the modified surface promotes adhesion of the modified surface to a plurality of adjacent surfaces.

In certain embodiments, the modified surface promotes adhesion of the modified surface to adjacent cells and tissue.

Also provided is a device comprising a surface and a functional layer associated with the surface, wherein the functional layer comprises monomeric particles having a structure substituted with a functional group, wherein the functional group is adapted to modify a property of the device, the device is sufficiently biocompatible for application to a multicellular organism, and the particles have an average diameter of about 5 nm to about 10 microns, provided that when the structure is silica, the functional group does not include an amino group.

Also provided is a device comprising a surface and a functional layer associated with the surface, wherein the functional layer comprises particles having a structure associated with a functional moiety, wherein the functional moiety is adapted to modify a property of the device, the device is sufficiently biocompatible for application to a multicellular organism, and the particles have an average diameter of about 5 nm to about 10 microns, provided that when the structure is an unsubstituted silica, the functional moiety does not include collagen.

In certain embodiments, the structure is non-covalently associated with the functional moiety.

In certain embodiments, the structure is substituted with a functional group such that the functional group is adapted to modify a property of the device and the functional group can be the same as or different from the functional moiety.

Further provided is an implantable device comprising a surface and a functional layer associated with the surface, wherein the functional layer comprises particles having a structure associated with a functional moiety, wherein the functional moiety is adapted to modify a property of the device, the device is sufficiently biocompatible for application to a multicellular organism, and the particles have an average diameter of about 5 nm to about 10 microns, provided that when the structure is unsubstituted silica, the functional moiety does not include collagen nor an amino group.

Also provided are methods of making the device of the invention. In one variant, the method of making the device of the invention comprises: providing a surface; and providing one or more functional layers on the surface, wherein at least one of the functional layers contains a functional group, such that a property of the device is modified by the functional group to provide the device. In another variant, the method of making the device of the invention comprises: providing a surface; and providing one or more functional layers on the surface, wherein at least one of the layers contains a functional moiety, such that a property of the device is modified by the functional moiety to provide the device.

### BRIEF DESCRIPTION OF SEVERAL VIEWS OF THE DRAWINGS

The invention will be described in conjunction with the following drawings in which like reference numerals designate like elements and wherein:

FIG. 1 is a flow chart of a method, pursuant to the invention, for preparing amine-terminated, colloidal silica and using it to coat a surface of a biomedical implant or other object.

FIG. 2 is schematic that provides greater detail for the “coating on substrate” box of the flow chart in FIG. 1.

FIG. 3 depicts data that characterize a type 316-L stainless steel surface modified with functionalized silica nanoparticles, pursuant to the present invention: (A) an atomic force-microscopic image, showing the presence of stacks of nanoparticles; (B) an EDAX spectrum of the surface, confirming the presence of functionalized silica nanoparticles.

FIG. 4 is an enlarged view of an atomic force microscope image of titanium surface coated with amino-functionalized silica nanoparticles further shown in FIG. 7(B).

FIG. 5 presents scanning electron micrographs of 316-L stainless steel and titanium surfaces seeded with MC3T3-E1 cells, wherein 316-L stainless steel surfaces were as follows: (A) unmodified surface; (B) SiO₂-modified; and (C) SiO₂—NH₂ modified and titanium surfaces were as follows: (D) unmodified surface; (E) SiO₂-modified; and (F) SiO₂—NH₂ modified.

FIG. 6 is a bar graph showing quantification of functional groups on modified metal surfaces. The abbreviations for a substrate surface used herein are as follows: Ti-titanium, SS-stainless steel, Ti+SiO₂— titanium and silicone oxide, SS+SiO₂ stainless steel and silicone oxide, Ti+SiO₂—NH₂ is titanium and silicone oxide substituted with an amino group, and SS+SiO₂—NH₂ is stainless steel and silicone oxide substituted with an amino group.

FIG. 7 presents atomic force micrographs that characterize titanium surfaces (A) unmodified and (B) modified with functionalized silica nanoparticles (FSNP) and polyurethane surfaces (C) unmodified and (D) modified with functionalized silica nanoparticles (FSNP).

FIG. 8 presents fluorescent images of DAPI stained MC3T3-E1 cells on (A) polyurethane surface, (B) polyurethane coated with SiO₂ nanoparticles, and (C) polyurethane coated with SiO₂ functionalized nanoparticles.

FIG. 9 presents scanning electron micrographs of titanium surface (A) unmodified and (B) modified with functionalized silica nanoparticle on a scale of 10 μm, (C) modified with functionalized silica nanoparticle on a scale of 100 nm, and (C) an EDAX spectrum of the surface, confirming the presence of functionalized silica nanoparticles.

### DETAILED DESCRIPTION OF THE INVENTION

The present invention flows from the discovery that functionalized particles, defined herein as particles having a structure substituted with a functional group, can be used to modify the topology and chemistry of various surfaces to provide a modified surface. The functionalized particles impart functionality to an unmodified surface, such that the resulting modified surface can accommodate cell attachment and development and/or impart to the functionalized surface chemical reactivity that is needed for the immobilization and presentation of bioactive molecules.

In the present invention, the functionality can be imparted to a surface by using a functional group, a functional moiety, or both in association with the structure. Such association can be covalent and/or non-covalent. As used herein, the expression “functional group” denotes a group covalently bonded to the structure, and the expression “functional moiety” denotes a group non-covalently associated with the structure.

Non-limiting examples of modification of surfaces used herein include modifications of cell behavior, modifications of a reaction to the surface of a cell of the multicellular organism, modifications of a reaction to the surface of a tissue of the multicellular organism, transfection of adjacent cells and tissue with genomic material, delivery of bioactive agents to adjacent cells and tissue, and promotion of adhesion of the modified surface to a plurality of adjacent surfaces including cells and tissue.

The surface modified by association with functional groups, functional moieties, or both is used in a device of the present invention or can be used as the device by itself.

The device of the invention comprises a surface and a functional layer associated with the surface, wherein the functional layer comprises particles having a structure substituted with a functional group, wherein the functional group is adapted to modify a property of the device, the device is sufficiently biocompatible for application to a multicellular organism and the particles have an average diameter of about 5 nm to about 10 microns.

In certain embodiments, the device is an implantable device. In another embodiment, the device is a drug delivery device. In yet another embodiment, the device is an outer surface contacting device adapted to contact an outer surface of the multicellular organism (non-limiting examples of which include, e.g., a topical bandage, a transdermal patch, etc.).

In certain embodiments, the multicellular organism is a human, an animal or a plant. Preferably, the multicellular organism is a human.

In certain embodiments, particles of the present invention are in the form of a modified colloidal dispersion, which comprises amine-terminated silica particles having a controlled size range, which are dispersed in an aqueous phase. When coupled with a diagnostic or therapeutic agent, the colloidal dispersion provides for the targeted delivery of such agents though transport channels and to the anatomical structures of interest. Suitable agents include, but are not limited to, imaging materials, targeting molecules or antibodies, biocompatible natural or synthetic molecules such as proteins and peptides, pharmaceuticals, and genetic materials.

The term “colloidal dispersion” as used herein refers to a stable dispersion of discrete particles, which are modified to carry at least one diagnostic or therapeutic agent and sized to remain suspended in an aqueous phase. Preferably, the colloidal dispersion comprises a particle-containing dispersion that can be applied, such as by pouring, spreading, painting, spraying, atomizing, injecting or inhaling, to reach a certain area or region, and that can subsequently change form by drying or gelation. Therefore, it includes the deposits, films, coatings (dense or porous), gels, droplets and aerosols derived from the colloidal dispersion. The colloidal dispersion of the present invention is particularly well suited to the targeted delivery of diagnostic and therapeutic agents to specific anatomical structures of a patient by coating or depositing on a surface. Non-limiting examples of particles useful in this invention are silica particles, polymer particles, metal and metal oxide particles.

The term “silica particles” as used herein refers to a plurality of discrete particles of the naturally occurring oxide of silicon having the approximate chemical formula SiO₂ without regard to shape, morphology, porosity, and water or hydroxyl content.

In certain embodiments, the functionalized particles of the invention can be applied onto a wide range of surfaces from aqueous solutions using various techniques known in the art, such as coating, spin-coating, dip-coating, painting, stamping, printing, or spraying. The functionalized particles of the invention can be used with various surfaces presented in all manner of configurations, such as a wire, a fiber, a filament, a coil, a tube, a sheet, a foil, a cylinder, a sphere, a mesh, a mat, a tube, a plate, a gel, or a hydrogel, which may be solid, hollow or porous. A coiled surface is especially useful in stent applications. Such a variety of approaches to deposit particles of the invention onto different surfaces allows a high degree of control over important surface properties, such as coating thickness, roughness, and the nature and density of chemical and biological functionalities.

The term “biological functionality” as used herein denotes a moiety or a group on a particle that is biologically active. Non-limiting examples of biological functionalities include a growth factor or bioactive polypeptide, or a polynucleotide coding therefor. Representative of this type of functionality are epidermal growth factor, acidic fibroblast growth factor, basic fibroblast growth factor, vascular endothelial growth factor, nerve growth factor, chondrogenic growth factor, platelet-derived growth factor, transforming growth factor beta, insulin-like growth factor, hepatocyte growth factor, bone morphogenic proteins, and osteogenic proteins. A biological functionality also can be a drug or drug-delivering agent, such as an antibody, a liposome, or a drug-delivery polymer.

Non-limiting examples of surfaces of the present invention include a metal, a metal oxide, silicon dioxide, a ceramic, a glass, a glass ceramic, a semiconductor, an amorphous insulator, a polymer, and a carbonaceous material.

In certain embodiments, when the surface is the metal, non limiting examples of the metal are aluminum, gold, silver, stainless steel, ferrous alloys, titanium, cobalt, nickel, mixtures thereof and alloys thereof.

Non-limiting examples of ceramic surfaces include alumina, zirconia, silica, magnesia, mullite, calcium phosphate, calcium silicate, calcium carbonate, mixtures thereof and alloys thereof.

Non-limiting examples of polymer surfaces include biodegradable polymers, non-biodegradable water-soluble polymers, non-biodegradable non-water soluble polymers, conductive polymers, and biopolymers. Preferably, the polymer is a member selected from the group consisting of polystyrene, polyurethane, polyethelene, polypropylene, poly(oxymethylene), polyacetal, poly(tetrafluroethyelene), silicone elastomer, polyvinylidine difluoride, polysulfone, and poly(methylmethacrylate). These polymers are especially useful in manufacturing orthopedic implants, sutures or surgical meshes. Also preferred are polymers selected from the group consisting of poly(pyrrole), poly(aniline), poly(thiophene), and poly(phenylene).

Non-limiting examples of the carbonaceous material include a pyrolytic carbon or a non-pyrolytic carbon.

The functionalized particles of the invention can be applied onto surfaces described above to form a functional layer.

The term “layer” includes a contiguous deposit of particles, non-contiguous deposit, single layer as well as multiple layers. Non-contiguous deposits can be produced in variety of shapes and forms, wherein particles are deposited as a pattern or randomly. Polymer on polymer stamping can also be used to form layers of the invention as described by Jiang, et al., Langmuir, 7, 2607-2615, Polymer-on-polymer stamping: Universal approaches to chemically patterned surfaces. An example of a patterned deposition is described by Xia et al. in Chemical Reviews 1999, 99, pp 1823-1848.

Preferably, the layer is highly homogeneous and high purity in that it consists essentially of functionalized particles.

Functional layers can include a mixture of different particles having the same functional group, or a mixture of particles having different functional groups. Also, layer(s) made of unmodified particles (particles without functional groups) can be applied in association with functional layer(s).

Also, functional layer(s) include functional moieties as defined further below. Such layers can be applied in association with layer(s) made of unmodified particles and/or functional layer(s) having functional groups.

One or more of these layers can be combined or conjugated with cells, polynucleotides, and/or pharmacologically active agents.

Particles of the Invention

Particles of the invention have a structure substituted with a functional group, wherein the functional group is adapted to modify a property of the device. The term “functionalized particles” and “particles” is used interchangeably to describe a structure substituted with a functional group.

The term “particle” as used herein denotes a size range that is about 5 nm to about 10 microns, preferably from 5 nm to about 1 micron. For purposes of this invention, therefore, the “microparticle” category includes very small to nanometer-sized particles.

The term “colloid” as used herein means a system in which finely divided particles (e.g., nanoparticles or particles of greater or lesser dimensions) are dispersed such that the particles are not easily filtered or precipitated rapidly and form a stable suspension.

The particles of the invention are substantially spherical and have a ratio of a major axis to a minor axis in a range of about 1.0 and to about 2.0. Preferably, the ratio is in a range of 1.0 to 1.2. In certain embodiments, the particles of the invention have polydispersity of less than 0.3, preferably the polydispersity is less than 0.1, and more preferably less than 0.01.

In certain embodiments, the structure of the particle of the invention is an inorganic molecule. The inorganic molecule is preferably selected from the group consisting of an oxide, a nitride, a carbide, calcium silicate, calcium phosphate, calcium carbonate, a carbonaceous material, a metal, and a semiconductor.

In certain embodiments of the invention when the structure is the metal, the metal is selected from the group consisting of aluminum, gold, silver, stainless steel, iron, titanium, cobalt, nickel, and alloys thereof.

In certain embodiments of the invention when the structure is the oxide, the oxide is selected from the group consisting of Al₂O₃, TiO₂, ZrO₂, Y₂O₃, ferric oxide, ferrous oxide, a rare earth metal oxide, a transitional metal oxide, SiO₂, mixtures thereof and alloys thereof. The oxides listed above can contain some additives or impurities, for example Al₂O₃ can contain some amounts of SiO₂ and MgO (or an alkaline earth oxide), and SiO₂ can contain some amounts of Na₂O (or an alkaline oxide), B₂O₃, and even P₂O₅.

In certain embodiments of the invention when the structure is the polymer, the polymer is selected from the group consisting of biodegradable polymers, non-biodegradable water-soluble polymers, non-biodegradable non-water soluble polymers, lipophilic moieties, and biopolymers. Preferably, the polymer is a member selected from the group consisting of polystyrene, polyurethane, polylactic acid, polyglycolic acid, polyester, poly(alpha-hydroxy acid), poly(ε-caprolactone), poly(dioxanone), poly(orthoester), poly(ether-ester), poly(lactone) mixtures thereof and copolymers thereof. These polymers are especially useful in sutures, meshes or tissue engineering scaffolds and drug delivery.

The functional group of the invention attached to the structure by methods known in the art. Functional groups may be affixed to colloids using a chemical (e.g., covalent) bond. For example, a silica surface can be reacted with a silane having a functional group such as amine. Functional groups may also be affixed to colloids using electrostatic attraction (e.g., a non-covalent association). For example, a negatively charged colloid surface can attract a positively charged functional group. It is also possible to combine the use of polymer and electrostatic attraction for affixing functional groups to the colloid surface. For example, a polyelectrolyte, which is polymer that can ionize and carry a charge, can be attracted to the oppositely charged colloidal surface. Such polyelectrolyte itself can carry a functional group. This process can be repeated many times. For example, after a positively charged polyelectrolyte A is absorbed to the surface of a colloid, a negatively charged polyelectrolyte B or simply another molecule B′ can be absorbed to the surface that is now positively charged due to the polyelectrolyte A's charge. Since polyelectrolyte A and B (or B′) may bear different functionalities, multi-functionality can also be incorporated.

Preferably, the functional group of the invention is a chemical functional group, a biomolecule, a photo-reactive moiety, or a photo-initiator moiety.

In certain embodiments, in which the functional group is the chemical functional group, it is selected from the group consisting of an amino group, a hydroxyl group, a carboxy group, a —SO₃H group, a —SH group, an —OCN group, a phosphorous group, an epoxy group, a vinylic moiety, a silane coupling agent, an acrylate, a methylacrylate, a metal alkoxy group, and derivatives thereof. In certain embodiments, when the structure is silica, the functional group does not include an amino group.

In certain embodiments, in which the functional group is the biomolecule, it is selected from the group consisting of a bioactive polypeptide, a polynucleotide coding for the bioactive polypeptide, a cell regulatory small molecule, a peptide, a protein, an oligonucleotide, adenoviral vectors, a gene transfection vector, a drug, and a drug delivering agent.

In certain embodiments, in which the functional group is the growth factor, it is selected from the group consisting of an epidermal growth factor, an acidic fibroblast growth factor, a basic fibroblast growth factor, a glial growth factor, a vascular endothelial growth factor, a nerve growth factor, a chondrogenic growth factor, a platelet-derived growth factor, a transforming growth factor beta, an insulin-like growth factor, a hepatocyte growth factor, bone morphogenic proteins and osteogenic proteins. Preferably, the bioactive polypeptide is a member selected from the group consisting of bone morphogenic proteins and osteogenic proteins.

Another way of imparting functionality to a surface and consequently to a device is by using a functional moiety.

In certain embodiments, the device of the invention comprises a surface and a functional layer associated with the surface, wherein the functional layer comprises particles having a structure associated with a functional moiety, wherein the functional moiety is adapted to modify a property of the device, the device is sufficiently biocompatible for application to a multicellular organism, and the particles have an average diameter of about 5 nm to about 10 microns, provided that when the structure is an unsubstituted silica, the functional moiety does not include collagen.

In certain embodiments, the device of the invention comprises a surface and a functional layer associated with the surface, wherein the functional layer comprises particles having a structure associated with a functional moiety, wherein the functional moiety is adapted to modify a property of the device, the device is sufficiently biocompatible for application to a multicellular organism, and the particles have an average diameter of about 5 nm to about 10 microns, provided that when the structure is unsubstituted silica, the functional moiety does not include collagen nor an amino group.

The functional moiety of the invention is non-covalently associated with the structure. Preferably, the functional moiety is a member selected from the group consisting of a growth factor, a bioactive polypeptide, a polynucleotide coding for the bioactive polypeptide, a cell regulatory small molecule, a peptide, a protein, an oligonucleotide, adenoviral vectors, a gene transfection vector, a drug, and a drug-delivering agent.

Preferably, the structure is an inorganic molecule selected from the group consisting of an oxide, a nitride, a carbide, calcium silicate, calcium phosphate, calcium carbonate, a carbonaceous material, a metal, and a semiconductor as defined above.

In one embodiment, the functionality is imparted by both a functional group and a functional moiety. In this case, the device having a functional moiety has the structure which is substituted with a functional group such that the functional group and a functional moiety are adapted to modify a property of the device and the functional group can be the same as or different from the functional moiety. Also, the modified property can be the same or different.

The functional group and the functional moiety as described above are used to modify at least one property of the device of the invention. Non-limiting examples of such properties include adhesion, friction, wettability, texture and roughness.

Also provided is a method of modifying a surface, said method comprising providing on the surface a functional layer comprising particles having a structure substituted with a functional group and/or associated with a functional moiety such that the functional layer modifies a property of the surface to provide a modified surface, wherein the modified surface is sufficiently biocompatible for application to a multicellular organism and the particles have an average diameter of about 5 nm to about 10 microns.

In certain variants of the method of modifying a surface, the modifications include modifications of cell behavior, modifications of a reaction to the surface of a cell of the multicellular organism, modifications of a reaction to the surface of a tissue of the multicellular organism, transfection of adjacent cells and tissue with genomic material, delivery of bioactive agents to adjacent cells and tissue, and promotion of adhesion of the modified surface to a plurality of adjacent surfaces including cells and tissue.

Also provided are methods of making the device of the invention. In one variant, the method of making the device of the invention comprises: providing a surface; and providing one or more functional layers on the surface, wherein at least one of the functional layers contains a functional group, such that a property of the device is modified by the functional group to provide the device. In another variant, the method of making the device of the invention comprises: providing a surface; and providing one or more functional layers on the surface, wherein at least one of the layers contains a functional moiety, such that a property of the device is modified by the functional moiety to provide the device.

Method of Making Particles Substituted with Functional Groups

The particles of the invention are preferably are in a colloidal preparation and made by any method suitable for obtaining a colloid, as described, for instance, by Morrison and Ross, COLLOIDAL DISPERSIONS: SUSPENSIONS, EMULSIONS AND FOAMS (Wiley Publ. 2002). Then, the preparation is chemically treated to obtain a modified surface and thereby imparting the desired functionality to the obtained modified surface.

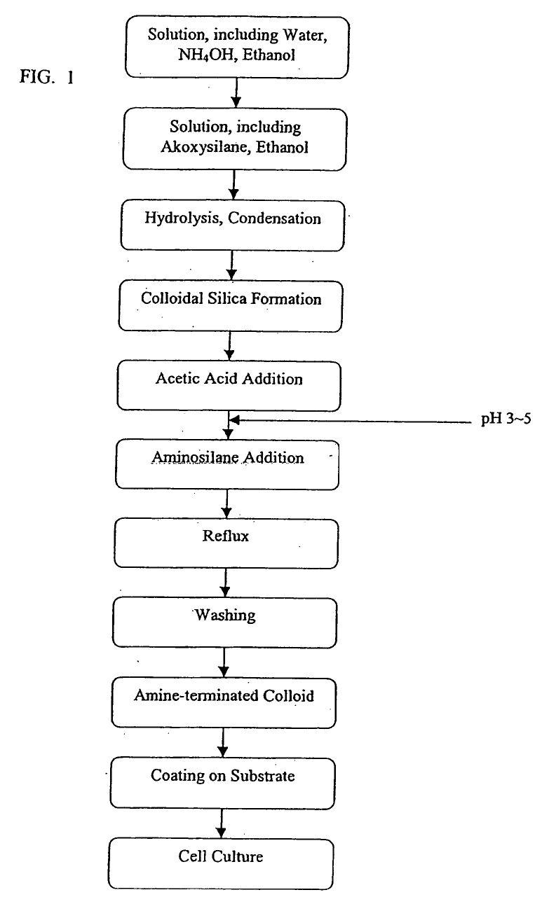

In one of the embodiments the invention, the particle is functionalized colloidal oxide, which can be prepared via a sol-gel process, functionalized, and then used for coating. By way of illustration, such particles are obtained by the condensation and hydrolysis of a metal oxide under acid or alkaline conditions from an alkoxy precursor (see FIG. 1). In this regard, the class of “metal oxides” includes Group IVA oxides, such as silica and germania.

The resultant colloidal particles of about 50 nm in diameter are modified before they are coated onto a surface as described above that is suitable for in vivo or in vitro contact with cells (i.e., is “cytocompatible”).

In general, particles can be functionalized with agents useful to make surfaces reactive, including coupling agents and adhesion promoters. Preferably, coupling agents and adhesion promoters are biocompatible. As shown in FIG. 1, for example, particles can be modified with a silane coupling agent, yielding an amine-functionalized silicon oxide colloid. Other coupling agents, suitable for this purpose, include an —OH, a —COOH, a —SO₃H, a —SH, or an epoxide functional group, or derivatives thereof.

Thus, to functionalize the particles, known silane-coupling agents can be used, as described, for example, in SILANE AND OTHER COUPLING AGENTS, pp.215-228 (1992). In general, such agents can be represented by Formula I:

(RO)₃Si —R′—Y (I)

where R is methyl, ethyl, and other alkyl groups, and so RO is an alkoxy group. The R groups can be the same or different. The silane can be reactive with moisture.

In Formula I, the R′ group is a bridging group that joins the reactive silane portion with the functional group Y. The bridging group can be, for example, a carbon-containing structure having 1-100, more particularly 2-50 and 2-10 carbon atoms. Preferred bridging groups are hydrocarbon and alkylene structures, and more preferably linear alkylene structures, such as —(CH₂)ₓ—, wherein X is 1-100, preferably 2-50, and more preferably 2-10.

In one embodiment of the invention, the bridging group R′ is non-reactive. In another embodiment, the bridging group itself can include one or more heteroatoms, such as nitrogen or oxygen or sulfur, apart from carbon and hydrogen, which can exist as an internal functional group different from functional group Y. Thus, the bridging group can comprise a secondary amine, or can include a tertiary amine structure. The bridging group can comprise a sulfide structure.

Y is a functional group that can be acidic, basic, or neutral. It can be ionic or nonionic, nucleophilic or electrophilic, a Lewis Acid or a Lewis Base. Y can be hydrophilic or hydrophobic, and it can have the capacity to hydrogen bond, whether as a donor or as an acceptor of hydrogen. Illustrative of Y are primary amine, secondary amine, tertiary amine, epoxide, hydroxyl, carbonyl-containing groups such as aldehyde, epoxy, carboxylic acid, and amide, sulfur groups including sulfonic acid, phosphorous groups including phosphonates and phosphates, thiols, and precursors and derivatives thereof.

With subsequently deposited layers, Y can provide for a chemical reaction, a charge interaction, or both. Also, Y can provide for formation of supramolecular structures, including host-guest complex formations, and/or can provide for self-assembly. Exemplary of chemical reactions for Y is the reaction of thiol group with gold or silver; reactions of hydroxyl and carbonyl groups (such as carboxylic acid, sulfonic acid, primary amine) with each other, resulting in the formation of ester, amide, anhydride, and imine (Schiff base) bonds, respectively. In addition, Y can be an epoxy, which will react, to open the epoxy ring, with any component such as amine, including primary and secondary amines, carboxylic acid, or hydroxyl. Examples of charge interactions for Y include primary amine ionic groups, such as sulfonic acid and carboxylic acid in electrolyte deposition. Illustrative of supermolecular complex formation is the avidin-biotin interaction. Examples of host-guest complex formation include cyclodextrin and steroids. In this context, self-assembly is illustrated by peptide dimerization, the formation of double-helix structures by complementary oligomers, and peptide insertion into another peptide.

Non-limiting examples of silane coupling agents include aminopropyltriethoxysilane, aminopropyltrimethoxysilane, aminopropylmethyldiethoxysilane, aminopropylmethyldimethoxysilane, aminoethylaminopropyltrimethoxysilane, aminoethylaminopropyltriethoxysilane, aminoethylaminopropylmethyldimethoxysilane, diethylenetriaminopropyltrimethoxysilane, diethylenetriaminopropyltriethoxysilane, diethylenetriaminopropylmethyldimethoxysilane, diethylenetriaminopropylmethyldiethoxysilane, cyclohexylaminopropyltrimethoxysilane, hexanediaminomehtyldiethoxysilane, anilinomethyltriethoxysilane, anilinomethyltrimethoxysilane, diethylaminomethyltriethoxysilane, (diethylaminomethyl)methyldiethoxysilane, methylaminopropyltrimethoxysilane, mercaptopropyltrimethoxysilane, mercaptopropyltriethoxysilane, bis(triethoxysilylpropyl)tetrasulfide, bis(triethoxysilylpropyl)disulfide, mercaptopropylmethyldimethoxysilane, 3-thiocyanatopropyltriethoxysilane, glycidoxypropyltrimethoxysilane, glycidoxypropyltriethoxysilane, glycidoxypropylmethyldiethoxysilane, glycidoxypropylmethyldimethoxysilane, methacryloxypropyltrimethoxysilane, methacryloxypropyltriethoxysilane, methacryloxypropylmethyldimethoxysilane, chloropropyltrimethoxysilane, chloropropyltriethoxysilane, chloromethyltriethoxysilane, chloromethyltrimethoxysilane, dichloromethyltriethoxysilane, vinyltrimethoxysilane, vinyltriethoxysilane, and vinyltris(2-methoxyethoxy)silane.

Formula I provides one silane group per molecule, but the molecule can comprise multiple silane groups, such as two, three, and four silane groups. In addition, non-silane coupling agents can be used including, for example, zirconium based coupling agents, zircoaluminates, and metal alkoxides.

In a preferred embodiment, the colloid is controlled to a pH in the range of 3 to 5, prior to surface modification, because silanol groups of aminosilane in aqueous solution are relatively stable in acid condition. Since silanol groups have an isoelectric point of about 2 to 3, the aminosilanes in the pH range of 3-6 must exist as zwitterions. The formation of zwitterions prevents the continuous hydrolysis and condensation of aminosilane.

In accordance with this embodiment of the invention, a water-stable, amine-terminated oxide is prepared by blocking consecutive reactions of aminosilane in aqueous condition. The amine-terminated oxide suspension is washed and stored, for later use as the source material for coatings.

A silica-modified surface of the present invention typically carries a film that is from about 200 to about 400 nm in thickness and is uncracked, adherent, wettable, and heat-resistant up to approximately 150° C. Moreover, such silica-modified surfaces have excellent cytocompatibility, and the introduction of amine functionalities, pursuant to the invention, significantly enhances cell proliferation.

Besides the above-described method of producing silica particles by the sol-gel method, any other suitable particles and particle production methods can be used instead.

Polymeric colloidal particles used in the invention can be typically prepared by methods known in the art and subsequently modified to attach functional groups or moieties as described above by using for example electrostatic attraction.

In the method of emulsification-solvent evaporation, the polymer is dissolved in chlorinated hydrocarbon (organic solvent) such as methylene chloride or chloroform as disclosed by Wise, Donald L. ed., Handbook of Pharmaceutical Controlled Release Technology, Marcel Dekker Incorporated, New York, N.Y., pages 329-344 (2000). The polymer solution is then mechanically dispersed in an aqueous solution containing a polymeric surfactant, such as polyvinyl alcohol (PVA) or carboxymethoxycellulose (CMC), by homogenization or ultrasonication to form a microemulsion. The thermodynamically unstable microemulsion is stabilized by the presence of PVA. The organic solvent is then evaporated and the colloids (and/or NPs) collected by centrifugation to remove the excess PVA and then resuspended in a solution of interest.

Niwa et al. have developed a method to produce polymeric colloidal particles by first dissolving the polymer in a mixture of chlorinated hydrocarbon such as methylene chloride and acetone, and then pouring this solution into an aqueous phase containing PVA with mechanical stirring. (See Controlled Rel., (25), 89-98 (1993)). Acetone is added to enhance the diffusion of the methylene chloride solvent into the water phase. Like the solvent evaporation approach the organic solvent is evaporated and the colloids are separated from the PVA phase by centrifugation. Their approach is called spontaneous emulsification solvent diffusion (SESD).

Murakami et al. have reported a modification of the SESD procedure that relies on the gelation of the PVA phase around the emulsion droplets for stabilization of the colloids as they form in solution. (See Intl. J. Pharm., (187), 143-152 (1999)). In this approach, to control and restrict the gelation of PVA to the surface of the emulsion droplet, alcohol (ethanol or methanol), which is a solvent for PVA but a non-solvent for the polymer was used. The mechanism of colloid formation is again dependent on the presence of the polymeric emulsifier, PVA. This method yields colloids of mean diameter of above 260 nm.

The particles of the invention may also include various ceramic (such as metal oxide or metal nitride) or metal particles, including but not limited to titania, zirconia, niobia, alumina, aluminum nitride, aluminum, gold and silver which are subsequently modified to attach functional groups or moieties as described above. Furthermore, the particles may be or include semiconductor particles, also known as “quantum dots,” composed of cadmium selenide, zinc selenide, cadmium telluride, and/or cadmium sulfide. Some of the quanum dots can be toxic to humans and would have to be used with caution or treated to mitigate toxicity.

Thus, a layer that is deposited, pursuant to the present invention, can be inorganic, including silica-based, or organic, including polymer colloids, such as polystyrene, PMMA, and degradable polymers, as well as metallic colloidal, and the like.

The particles also can be produced by mechanical processing, physical (thermal) processing or by chemical processing other than the sol-gel method. In mechanical processes, fine powders are commonly made from large particles using crushing techniques such as a high-speed ball mill. In chemical processes other than the sol-gel process, nanocrystalline materials are created from a reaction that precipitates particles of varying sizes and shapes, using a family of materials known as organometallics (substances containing combinations of carbon and metals, bonded together), inorganic salts, or their mixtures. Alternatively, particles may be formed by combustion processes by passing a mist of dissolved organic or inorganic salt through a high temperature zone, optionally with the aid of another combustant, to cause rapid conversion.

Physical or thermal processes involve the formation and collection of particles through the rapid cooling of a supersaturated vapor (gas phase condensation), as described in U.S. Pat. No. 5,128,081 and U.S. Published Application 2002/0053557. Physical or thermal processes create the supersaturated vapor in a variety of ways, including laser ablation, plasma torch synthesis, combustion flame, exploding wires, spark erosion, electron beam evaporation, sputtering (ion collision), and electrothermal gun or “electrogun” methodology, as described in the aforementioned published application.

In the physical process described in U.S. Pat. No. 5,128,081, a raw metallic material is evaporated into a chamber and raised to very high temperatures, and then oxygen is rapidly introduced. A carrier medium may also be mixed with precursor material which is vaporized and subsequently rapidly quenched, as disclosed in U.S. Pat. No. 5,851,507.

In laser ablation, a high-energy pulsed laser is focused on a target containing the material to be processed. The high temperature of the resulting plasma (greater than 10,000° K) vaporizes the material so quickly that the rest of the source (any carrier and quenching gases) can operate at room temperature.

In the combustion flame and plasma torch processes the precursor material can be a solid, liquid or gas prior to injection into the flame or torch, under ambient pressure conditions (the most common precursor state is a solid material). The primary difference between the two processes is that the combustion flame involves the use of an oxidizing or reducing atmosphere, while the plasma torch uses an inert gas atmosphere, as described in U.S. Pat. Nos. 5,876,683, 4,642,207 and 5,486,675. Alternatively, the feed material may be delivered to the plasma stream by arc vaporization of the anode. The anode is normally metallic but may be a metal-ceramic composite. Another plasma torch can produce non-agglomerated ceramic nanocrystalline powders starting from metallorganic precursors, and uses rapid thermal decomposition of a precursor/carrier gas stream in a hot tubular reactor combined with rapid condensation of the product particle species on a cold substrate, as described in U.S. Pat. No. 5,514,349.

In the electrogun process, a pulsed, high current arc is struck down the barrel of a gun, between an electrode in the breech of the gun and an electrode at the muzzle. This arc produces rapid vaporization of the electrodes and of the barrel of the gun so that a pulsed, high-temperature, high-velocity plasma jet is fired out the muzzle.

Semiconductor particles often are made by a chemical organometallic method. In one such method, an organometallic raw material, such as Cd(CH₃)₂ decomposes in a hot solvent, such as TOPO. When a strong ligand, such as HPA or TDPA, is added to the solvent, Cd(CH₃)₂ is converted to a Cd(CH₃)₂/HPA or TDPA complex. An injection of selenium dissolved in TBP into the solution generates CdSe particles. The particles are then placed into an organic solvent, such as toluene, as described in Peng, J. Am. Chem. 2001: 123183, and U.S. Pat. No. 6,207,229. Alternatively, the particles may be made by the so-called “core/shell” method, where one type of semiconductor nanoparticle, such as CdSe is encapsulated in a shell of another semiconductor material, such as ZnSe, or by a surfactant method, as described in U.S. Pat. No. 6,225,198. Any other suitable chemical method also may be employed.

Metal and ceramic particles made by physical processes are commercially available from Nanotechnologies, Inc. (Austin, Tex.), for example. Semiconductor particles made by the chemical process are available from Evident Technologies (Troy, N.Y.), under the brand name EVIDOTS™. Polymer microparticles are usually made by an emulsion technique and are commercially available from Duke Scientific Corporation (Palo Alto, Calif.), for example.

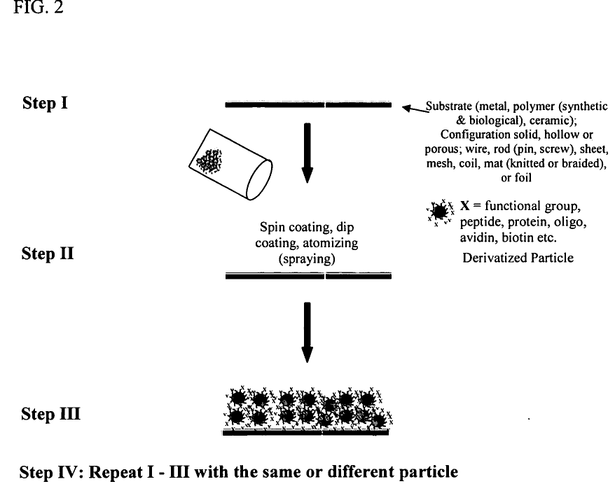

Once particles are treated to contain functional groups, they are applied to various surfaces described above. In a preferred embodiment, the particles are coated onto a surface to form a layer. The steps of a coating process that is illustrative of the present invention are shown in FIG. 2. To prepare for coating, the chosen surface preferably first is cleaned, in appropriate solutions. The functionalized particles then are applied to the surface, by any of a variety of conventional techniques that can afford well-controlled coverage of the given surface. Such techniques include, for example, spin-coating, dip-coating, painting, stamping, printing, and spraying (atomizing). The resultant coating then is dried at a temperature, typically in the range of 60° to 120° C., that should not be so high or for such duration as to affect adversely the surface functionalities, by causing additional chemical reactions or removal of chemical groups. The result is a highly homogeneous, high-purity film.

Pursuant to the invention, functionalized particles preferably are applied to a surface of a biomedical implant, such as a ceramic or metal implant. The particles also can be put on a semiconductor or amorphous insulator surface. The latter can be, for example, a surface of a semiconductor device that serves as a biosensor or chemical sensor, such as a Chemically Sensitive Field-Effect Transitor (ChemFET) or a microcantilever sensor. In this regard, the semiconductor material may be silicon, while the amorphous insulator material may be a silicon nitride or silicon dioxide layer. These materials frequently are used in ChemFETs and microcantilever sensors. Preferably, the particles are coated on a surface of a sensor that will be implanted into living tissue, such as human or animal tissue. Illustrative sensors of this sort include ChemFET arrays, microcantilever sensors, and photodetectors and photodiodes, i.e., devices that perform optical measurements in vivo.

### EXAMPLES

The invention will be illustrated in more detail with reference to the following Examples, but it should be understood that the present invention is not deemed to be limited thereto.

### Example 1

0.05 mm-thick foils of type 316-L stainless steel and titanium (99.99%) were obtained from Goodfellow Corporation (UK). For coating studies, the foils were cut into rectangle pieces (0.7 mm×10 mm), were cleaned ultrasonically in hexane and acetone for 5 minutes each and subsequently washed in ethanol, and then were rinsed in deionized water, followed by drying at 60° C. oven temperature for a 24-hour period.

A mono-dispersed, nano-particulate silica colloid was prepared by the so-called Stober process, see SOL-GEL SCIENCE: THE PHYSICS AND CHEMISTRY OF SOL-GEL PROCESSING (Academic Press 1990), using tetraethylorthosilicate (TEOS), Si(OC₂H₅)₄, as the silica precursor and ammonia and alcohol as catalysts. The first step of this process entailed the hydrolysis of TEOS to the hydroxyl, intermediately followed by condensation of silicic acid, yielding silicon oxide (SiO₂). Thereafter, the surface of the colloidal particles was modified to bear amine groups by reacting the colloid with a silane coupling agent, aminopropyltriethoxy-silane (APS), H₂N(CH₂)₃Si(OCH₃)₃.

Laser light-scattering studies revealed the amine-bearing colloidal silica particles to be highly uniform and approximately 50 nm in diameter. The particles were deposited onto the cleaned foil specimens by spin-coating (2000 rpm, 20 seconds/layer, 10 layers) from an ethanolic solution. Between successive coatings, the surface was dried for 5 minutes, to ensure evaporation of ethanol. Coated substrates were dried for a 24-hour period before oven drying at 60° C. for another 24-hour period.

The modified surfaces of the stainless steel and titanium foils were characterized by means of scanning electron microscopy, X-ray photoelectron spectroscopy, energy dispersive X-ray analysis, Rutherford back scattering, and atomic force microscopy (AFM, tapping mode image):

Scanning Electron Microscopy (SEM) allows for micron-range examination of surface characteristics. SEM images were obtained on the JOEL 6300FV microscope, at an accelerating voltage of 10 kV and at a vacuum pressure of 3 E-6 Torr (4E-4 Pa) During SEM analysis, the back scattered electrons were used to determine the elemental composition of the surface by EDAX.

Atomic Force Microscopy (AFM) enables a contour mapping of a surface, providing information about various aspects of surface topology, such as direct height measurements and friction imaging. AFM images were obtained on Dimension 3100 Series (Digital Instrument) in tapping mode, using a prefabricated cantilever. Topographical results were viewed in two- and three-dimensional representations, and topographical data were collected.

Rutherford Back-Scattering (RBS) was employed to determine film thickness on the substrates. RBS uses elastic scattering of 0.1-3 MeV charged particles to analyze the surface and the outer few micrometers of solids. Thus, samples were bombarded with 0.1-3 MeV protons or alpha particles from a Van de Graaff electrostatic accelerator, and a surface barrier detector detected the scattered particles. The signal from the detector was processed by common nuclear electronics, and the particle energy spectra were stored in a computer-based multi-channel analyzer. The data evaluation was effected with standard procedures and computer codes. When a positively charged (He atoms) ion beam (2.023 MeV and 20 nA) was directed at each sample, in a vacuum chamber, the average thickness of the SiO₂ colloid coating was 380 nm for the stainless steel samples and 410 nm for the titanium samples.

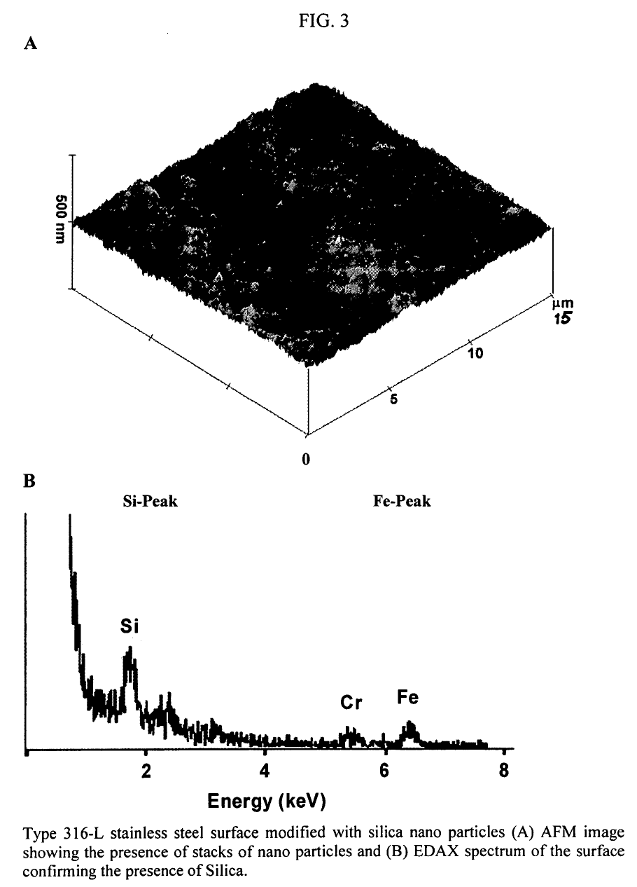

The silica nano-particles were obtained as a mono-dispersed preparation, with a mean diameter of 58 nm, ascertained by dynamic laser light scattering. The formation of an amine-rich surface upon derivatization with APS was confirmed by a salisaldehyde-based calorimetric assay at 404 nm. The presence of silica nano-particles on the metal surface was confirmed by AFM and EDAX analysis (FIGS. 3A and B, respectively). RBS analysis showed that the thickness of the silica-nanoparticle layer was reproducible and ranged from 380-420 nm, depending on the metal substrate, for a 10-layer coating.

### Example 2

Cell-attachment and proliferation studies were carried out on the sample prepared in Example 1, using MC3T3-E1 osteoblast-like cells. Cells were cultured in alpha-MEM supplemented with 10% FBS and 1% pen-strep under standard culture conditions. Metal substrates were mounted in 4-chambered glass slides, using sterile vacuum grease (Dow Corning), and then were surrounded with low-melting agarose (1% solution), to prevent cell attachment to the glass surface. Cells were seeded at 10,000 cells/well (1 ml). After four days the medium was aspirated, and the cells were trypsinized and then manually counted with a hemacytometer. Samples for SEM analysis were prepared by first fixing with 1% glutaraldehyde followed by dehydration in an ethanol series (50%, 60%, 80%, 90%).

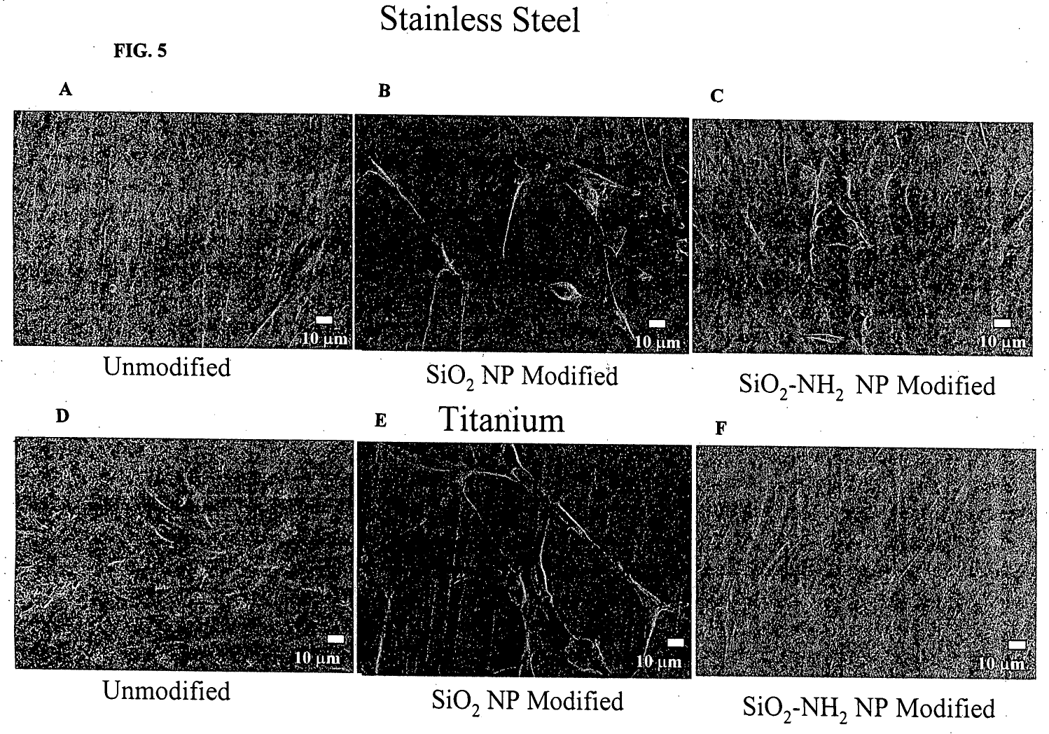

These studies showed that the aminopropyltriethoxy-silane modified silica surfaces had excellent cytocompatibility. The presence of a modified surface did not diminish cell attachment, relative to unmodified metal surfaces. On the contrary, the introduction of an amine-modified surface significantly enhanced cell proliferation as discussed in Example 4 and shown in Table 1. This result was consistent with SEM observations, where increased cell attachment and spreading was observed in the order: unmodified metal<metal-SiO₂<metal-SiO₂—NH₂ (FIG. 5).

### Example 3

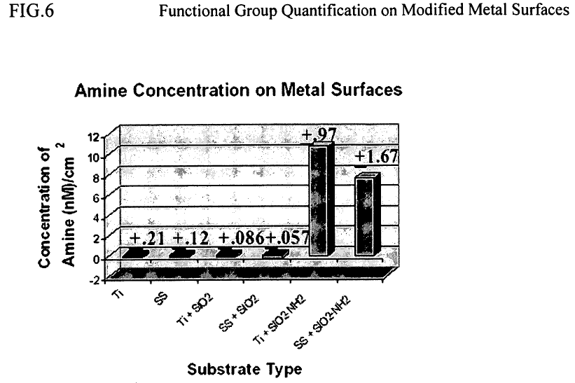

As shown in FIG. 6, nominal concentration of amine groups that are amenable to functionalization reactions were identified using an assay described by Gaur et al. (Analytical Biochemistry, 1989, 180, 243). The assay involves first reacting the amine group with sulfo-SDTB followed by hydrolysis of the adduct with perchloric acid to liberate a colored cation that is assayed spectrophotometrically at 498 nm.

Surface amine concentration was calculated as follows:

Ti+SiO₂—NH₂ particle=0.65 NH₂ groups/Å²

SS+SiO₂—NH₂ particle=0.46 NH₂ groups/Å²

### Example 4

Cell proliferation studies were carried out using MC3T3-E1 osteoblast-like cells. All cell culture studies were carried out at 37° C. in 5% CO₂ in a humidified incubator. Metal substrates were mounted in 4-chambered glass slides using sterile vacuum grease. Low-melting agarose (1% solution) was placed around the metals to prevent cell attachment to the glass surface. Cells were seeded at 10,000 cells/well (1 mL) and on the fourth day were counted using a hemacytometer.

Average cell counts for each substrate were tested. A total of seven substrates per sample type were evaluated. NP is abbreviation for a nanoparticle. Results are represented in Table 1 below.

TABLE 1
Average cell count

| | Average Cell Count |
|---|---|
| Sample Type | (10⁴ cells/cm²) |
| Tissue Culture Polystyrene (TCPS) | 5.77 ± 0.53 |
| Agarose | 0 |
| Titanium | 3.28 ± 1.4 |
| Titanium + SiO₂NP | 3.59 ± 1.4 |
| Titanium + SiO₂—NH₂NP | 7.43 ± 1.2 |
| Stainless Steel | 1.89 ± 0.89 |
| Stainless Steel + SiO₂NP | 3.80 ± 0.75 |
| Stainless Steel + SiO₂—NH₂NP | 5.48 ± 0.78 |

Cell proliferation on silica (SiO₂) coated titanium substrates was comparable to unmodified metal substrates. The introduction of the amine rich nano-particle (SiO₂—NH₂ NP) enhanced proliferation to a level comparable to or exceeding that of TCPS.

### Example 5

A comparison between metal and polymer surfaces is shown below in Table 2.

TABLE 2
Average film thickness and roughness
data for metal and polymer substrates.

| | Average Film | Average Film |
|---|---|---|
| | Thickness | Roughness |
| | (Rutherford Back | (Atomic Force |
| Sample Type | Scattering); nm | Micropscopy); nm |
| Titanium | N/A | 51.4 |
| Titanium + | 380 | 21.1 |
| SiO₂—NH₂NP | | |
| Stainless Steel | N/A | 31.0 |
| Stainless Steel + | 410 | 27.3 |
| SiO₂—NH₂NP | | |
| Polyurethane | N/A | 3.1 |
| Polyurethane + | N/A | 27.4 |
| SiO₂NP | | |
| Polyurethane + | N/A | 31.8 |
| SiO₂—NH₂NP | | |

The area scanned for roughness measurements for the metals was ˜51 μm². For the polyurethane samples, it was ˜10 μm².

Nanoparticle coatings were analyzed using atomic force microscopy (AFM) and Rutherford back-scattering (RBS). Coating roughness was measured using AFM and coating thickness was measured using RBS. A smoothening of the metal surface resulted after the introduction of a nanoparticle coating. The roughness of the nanoparticle coated surfaces was comparable in both the metal and the polymer substrates and reflected the underlying texture of the nanoparticles (50-60 nm in diameter). Substrates coated with the nanoparticles resulted in films of approximately the same thickness.

### Example 6

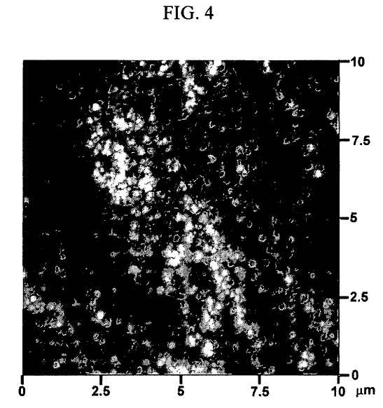

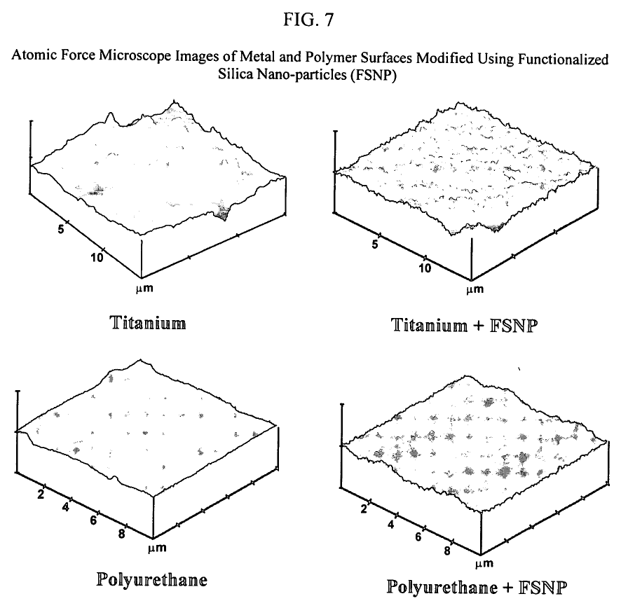

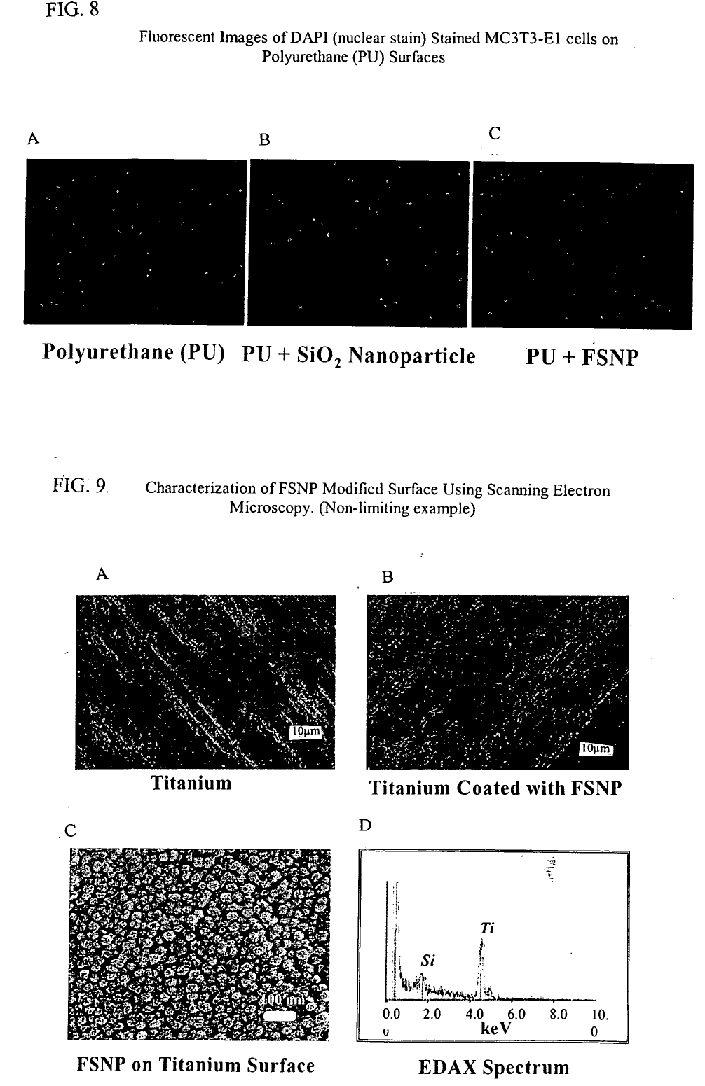

Metal substrates were cut into rectangular pieces (0.7 mm×10 mm), ultrasonically cleaned in hexane and acetone for 5 minutes each, subsequently washed in ethanol, and followed by rinsing in deionized water and dried in a 60° C. oven for 24 hours prior to use. Modification of metal substrates with nanoparticles was carried out by spin coating. Samples were mounted on the spin coater stage and held in place by light vacuum. Nanoparticle solution (50 μL, 1 wt %) was dropped onto substrate (spun at 2000 rpm for 20 seconds) using a pipette. Subsequent layers were deposited at 1-2 minutes intervals for a total often depositions. Samples were left to dry overnight and were heat treated at 80° C. for two hours. Polymer substrates were prepared as follows. Polyurethane was dissolved in tetrahydrofuran (THF) to make a 1 wt % solution. Polyurethane films were cast onto glass slides (10 mm×10 mm) that were cleaned by the aforementioned procedure using spin coating. Two coatings (150 μL, 300 rpm; 25 seconds) of polyurethane were deposited onto glass substrates with 1-2 minute intervals between coatings. The polyurethane solution was kept in a warm bath (37° C.) to avoid polymer precipitation when casting the films. Polyurethane coated substrates were dried overnight before nanoparticle deposition. Nanoparticles were deposited onto the polyurethane films using the same spin coating procedure outlined above. Samples were allowed to dry overnight and were heat treated on a hot plate (40° C.) for 15 minutes. Surfaces were characterized using atomic force microscopy (AFM) for roughness and film homogeneity as shown in FIGS. 7 and 4.

The initially rough titanium surface became smoother after nanoparticle deposition and surface texture reflected that of the nanoparticles (See FIG. 4). The initially smooth polyurethane film became rougher after coating and the roughness reflected the texture of the nanoparticles. However, both metal and polymer substrates after nanoparticle modification had similar surface roughness data.

### Example 7

### Patterned Assembly of FSNP

Patterned assembly of FSNP was performed using the following procedure:

1. Introduce lipophilic/hydrophilic regions via Micro-contact printing, Ink Jet printing or by selective masking onto substrate of choice;

2. Introduce FSNP of choice;

3. Build NP Layers using next FSNP of choice—leverage electrostatic, hydrophobic, lipophilic interactions to create 3D hierarchical structures; and

4. Removal of “masking” substance.

A glass slide was ultrasonically cleaned sequentially in methylene chloride, hexane, and acetone followed by distilled deionized water. A polydimethysiloxane elastomer (PDMS) stamp was then inked with polystyrene latex particles labeled with Rhodamine dye, brought in contact with the glass surface, and gently pressed against the surface for couple of seconds. The stamp was removed, and the Rhodamine labeled polystyrene (PS) particles were transferred.

Alternatively, the patterned surface can be achieved by first selectively blocking certain areas on a surface with a material that repels colloid adsorption and then exposing the surface to the colloidal solution. In the former case a positive stamp is used and in the latter case a negative stamp is used, wherein negative is with respect to the desired pattern.

The FSNPs were assembled on the polyurethane (PU) film as in the case of the metal substrates, by spin coating. The PU film substrates were prepared by spin casting of PU in THF onto a glass slides as described above. The MC-3T3-E1 attachment studies were also carried out as described in Example 2.

### Example 8

This example demonstrates making a functional layer by using a functional moiety/particle combination.

First, the functional moiety as described above, e.g., collagen, is deposited on particles to form particles functionalized with the moiety, e.g., “collagenated” particles. Then, a surface is coated with resulted “collagenated” particles to form a homogeneous layer. Next, “collagenated” particles can be applied repeatedly to build up a layer of required thickness from about 5 nm to 10 μm or higher.

Functional moieties can be of one kind of molecules or a mixture of different molecules. Particles can also be of one kind of structure or a mix of different structures. Also, particles are preferably modified with functional groups as described above. Moreover, functional moieties and functional groups can be of the same kind of molecules, e.g, biomolecules but attached to particles by different mechanism, e.g., non-covalent and covalent bonding.

While the invention has been described in detail and with reference to specific examples thereof, it will be apparent to one skilled in the art that various changes and modifications can be made therein without departing from the spirit and scope thereof.

What is claimed is:

1. A device comprising a surface and a functional layer associated with the surface, wherein the functional layer comprises particles having a structure substituted with a functional group, wherein the functional group is adapted to modify a property of the device, the device is sufficiently biocompatible for application to a multicellular organism and the particles have an average diameter of about 5 nm to about 10 microns.

2. The device of claim 1, wherein the device is an implantable device.

3. The device of claim 1, wherein the device is a drug delivery device.

4. The device of claim 1, wherein the device is an outer surface contacting device adapted to contact an outer surface of the multicellular organism.

5. The device of claim 1, wherein the multicellular organism is a human.

6. The device of claim 1, wherein the surface is a member selected from the group consisting of a metal, a metal oxide, silicon dioxide, a ceramic, a glass, a glass ceramic, a polymer, and a carbonaceous material.

7. The device of claim 6, wherein the metal is a member selected from the group consisting of aluminum, gold, silver, stainless steel, ferrous alloys, titanium, cobalt, nickel, mixtures thereof and alloys thereof.

8. The device of claim 6, wherein the ceramic is a member selected from the group consisting alumina, zirconia, silica, magnesia, mullite, calcium phosphate, calcium silicate, calcium carbonate, mixtures thereof and alloys thereof.

9. The device of claim 6, wherein the polymer is a member selected from the group consisting of biodegradable polymers, non-biodegradable water-soluble polymers, non-biodegradable non-water soluble polymers, conductive polymers, and biopolymers.

10. The device of claim 9, wherein the polymer is a member selected from the group consisting of polystyrene, polyurethane, polyethelene, polypropylene, poly(oxymethylene), polyacetal, poly(tetrafluroethyelene), silicone elastomer, polyvinylidine difluoride, polysulfone, and poly(methylmethacrylate).

11. The device of claim 9, wherein the polymer is a member selected from the group consisting of poly(pyrrole), poly(aniline), poly(thiophene), and poly(phenylene).

12. The device of claim 6, wherein the carbonaceous material is a pyrolytic carbon or a non-pyrolytic carbon.

13. The device of claim 1, wherein the surface is a member selected from the group consisting of a fiber, a filament, a coil, a tube, a sheet, a foil, a cylinder, a sphere, a mesh, a mat, a gel, and a hydrogel.

14. The device of claim 1, wherein the average diameter of particles is from 5 nm to 1 micron.

15. The device of claim 1, wherein the particles are substantially spherical and have a ratio of a major axis to a minor axis in a range of about 1.0 and to about 2.0.

16. The device of claim 15, wherein the ratio is in a range of 1.0 to 1.2.

17. The device of claim 1, wherein the particles have a polydispersity of less than 0.3.

18. The device of claim 17, wherein the polydispersity is less than 0.1.

19. The device of claim 17, wherein the polydispersity is less than 0.01.

20. The device of claim 1, wherein the structure is an inorganic molecule selected from the group consisting of an oxide, a nitride, a carbide, calcium silicate, calcium phosphate, calcium carbonate, a carbonaceous material, a metal, and a semiconductor.

21. The device of claim 20, wherein the metal is a member selected from the group consisting of aluminum, gold, silver, stainless steel, iron, titanium, cobalt, nickel, and alloys thereof.

22. The device of claim 20, wherein the oxide is a member selected from the group consisting of Al₂O₃, TiO₂, ZrO₂, Y₂O₃, ferric oxide, ferrous oxide, a rare earth metal oxide, a transitional metal oxide, SiO₂, mixtures thereof and alloys thereof.

23. The device of claim 1, wherein the structure is a polymer, and the polymer is a member selected from the group consisting of biodegradable polymers, non-biodegradable water-soluble polymers, non-biodegradable non-water soluble polymers, lipophilic moieties, and biopolymers.

24. The device of claim 23, wherein the polymer is a member selected from the group consisting of polystyrene, polyurethane, polylactic acid, polyglycolic acid, polyester, poly(alpha-hydroxy acid), poly(ε-caprolactone), poly(dioxanone), poly(orthoester), poly(ether-ester), poly(lactone) mixtures thereof and copolymers thereof.

25. The device of claim 1, wherein the functional group is a member selected from the group consisting of a chemical functional group, a biomolecule, a photo-reactive moiety, and a photo-initiator moiety.

26. The device of claim 25, wherein the chemical functional group is a member selected from the group consisting of an amino group, a hydroxyl group, a carboxy group, a —SO₃H group, a —SH group, an —OCN group, a phosphorous group, an epoxy group, a vinylic moiety, a silane coupling agent, an acrylate, a methylacrylate, a metal alkoxy group, and derivatives thereof.

27. The device of claim 25, wherein when the structure is silica, the functional group does not include an amino group.

28. The device of claim 25, wherein the biomolecule is a member selected from the group consisting of a bioactive polypeptide, a polynucleotide coding for the bioactive polypeptide, a cell regulatory small molecule, a peptide, a protein, an oligonucleotide, adenoviral vectors, a gene transfection vector, a drug, and a drug delivering agent.

29. The device of claim 28, wherein the bioactive polypeptide is a growth factor and such growth factor is a member selected from the group consisting of an epidermal growth factor, an acidic fibroblast growth factor, a basic fibroblast growth factor, a glial growth factor, a vascular endothelial growth factor, a nerve growth factor, a chondrogenic growth factor, a platelet-derived growth factor, a transforming growth factor beta, an insulin-like growth factor, a hepatocyte growth factor, bone morphogenic proteins and osteogenic proteins.

30. The device of claim 29, wherein such growth factor is a member selected from the group consisting of bone morphogenic proteins and osteogenic proteins.

31. The device of claim 1, wherein the property is a member selected from the group consisting of adhesion, friction, wettability, texture and roughness.

32. A method of modifying a surface, said method comprising providing on the surface a functional layer comprising particles having a structure substituted with a functional group and/or associated with a functional moiety such that the functional layer modifies a property of the surface to provide a modified surface, wherein the modified surface is sufficiently biocompatible for application to a multicellular organism and the particles have an average diameter of about 5 nm to about 10 microns.

33. The method of claim 32, wherein the property is a member selected from the group consisting of adhesion, friction, wettability, texture and roughness.

34. The method of claim 32, wherein the functional layer modifies a reaction to the surface of a cell of the multicellular organism.

35. The method of claim 32, wherein the functional layer modifies a reaction to the surface of a tissue of the multicellular organism.

36. The method of claim 32, wherein the modified surface transfects with genomic material adjacent cells and tissue.

37. The method of claim 32, wherein the modified surface delivers bioactive agents to adjacent cells and tissue.

38. The method of claim 32, wherein the modified surface promotes adhesion of the modified surface to a plurality of adjacent surfaces.

39. The method of claim 32, wherein the modified surface promotes adhesion of the modified surface to adjacent cells and tissue.

40. A device comprising a surface and a functional layer associated with the surface, wherein the functional layer comprises monomeric particles having a structure substituted with a functional group, wherein the functional group is adapted to modify a property of the device, the device is sufficiently biocompatible for application to a multicellular organism, and the particles have an average diameter of about 5 nm to about 10 microns, provided that when the structure is silica, the functional group does not include an amino group.

41. A device comprising a surface and a functional layer associated with the surface, wherein the functional layer comprises particles having a structure associated with a functional moiety, wherein the functional moiety is adapted to modify a property of the device, the device is sufficiently biocompatible for application to a multicellular organism, and the particles have an average diameter of about 5 nm to about 10 microns, provided that when the structure is an unsubstituted silica, the functional moiety does not include collagen.

42. The device of claim 41, wherein the structure is non-covalently associated with the functional moiety.

43. The device of claim 41, wherein the functional moiety is a member selected from the group consisting of a growth factor, a bioactive polypeptide, a polynucleotide coding for the bioactive polypeptide, a cell regulatory small molecule, a peptide, a protein, an oligonucleotide, adenoviral vectors, a gene transfection vector, a drug, and a drug delivering agent.

44. The device of claim 41, wherein the structure is an inorganic molecule selected from the group consisting of an oxide, a nitride, a carbide, calcium silicate, calcium phosphate, calcium carbonate, a carbonaceous material, a metal, and a semiconductor.

45. The device of claim 44, wherein the metal is a member selected from the group consisting of aluminum, gold, silver, stainless steel, iron, titanium, cobalt, nickel, and alloys thereof.

46. The device of claim 44, wherein the oxide is a member selected from the group consisting of Al₂O₃, TiO₂, ZrO₂, Y₂O₃, ferric oxide, ferrous oxide, a rare earth metal oxide, a transitional metal oxide, SiO₂, mixtures thereof and alloys thereof.

47. The device of claim 41, wherein the structure is a polymer, and the polymer is a member selected from the group consisting of biodegradable polymers, non-biodegradable water-soluble polymers, non-biodegradable non-water soluble polymers, lipophilic moieties, and biopolymers.

48. The device of claim 41, wherein the structure is substituted with a functional group such that the functional group is adapted to modify a property of the device and the functional group can be the same as or different from the functional moiety.

49. The device of claim 48, wherein the functional group is a member selected from the group consisting of a chemical functional group, a biomolecule, a photo-reactive moiety, and a photo-initiator moiety.

50. An implantable device comprising a surface and a functional layer associated with the surface, wherein the functional layer comprises particles having a structure associated with a functional moiety, wherein the functional moiety is adapted to modify a property of the device, the device is sufficiently biocompatible for application to a multicellular organism, and the particles have an average diameter of about 5 nm to about 10 microns, provided that when the structure is unsubstituted silica, the functional moiety does not include collagen nor an amino group.

51. A method of making the device of claim 1, comprising:
providing a surface; and
providing one or more functional layers on the surface, wherein at least one of the functional layers contains a functional group, such that a property of the device is modified by the functional group to provide the device.

52. A method of making the device of claim 41, comprising:
providing a surface; and
providing one or more functional layers on the surface, wherein at least one of the layers contains a functional moiety, such that a property of the device is modified by the functional moiety to provide the device.
